(19) United States
(12) Patent Application Publication
(10) Pub. No.: US 2023/0190878 A1
(43) Pub. Date: Jun. 22, 2023

(54) BETA THYMOSIN PEPTIDES FOR TREATING VIRAL INFECTIONS

(71) Applicant: REGENERX BIOPHARMACEUTICALS, INC., Rockville, MD (US)

(72) Inventors: Allan L. GOLDSTEIN, Rockville, MD (US); Gabriel SOSNE, Rockville, MD (US); Jack FINKELSTEIN, Jr., Rockville, MD (US)

(73) Assignee: REGENERX BIOPHARMACEUTICALS, INC., Rockville, MD (US)

(21) Appl. No.: 17/997,212

(22) PCT Filed: May 3, 2021
(86) PCT No.: PCT/US2021/030472

§ 371 Date: Oct. 26, 2022

Related U.S. Application Data
(60) Provisional application No. 63/020,345, filed on May 5, 2020.
(60) Provisional application No. 63/078,436, filed on Sep. 15, 2020.

(51) Int. Cl. A61K 38/22

(52) U.S. Cl. CPC A61K 38/2292

(57) ABSTRACT

A method of treating a viral respiratory infection, such as infection by at least one of a SARS virus, a coronavirus or SARS-CoV-2, includes administering to a subject in need of such treatment an effective amount of a composition including a peptide agent which includes a beta thymosin peptide with amino acid sequence LKKTET or LKKTNT, a conservative variant thereof, or a stimulating agent that stimulates production of an LKKTET or LKKTNT peptide, or a conservative variant thereof.

### BACKGROUND OF THE INVENTION

### Field of the Invention

The present invention relates to the field of treating viral infections.

### Description of the Background Art

There remains a need in the art for methods of treating infections caused by a SARS virus, a coronavirus and/or SARS-CoV-2.

### SUMMARY OF THE INVENTION

In accordance with one aspect, a method of treating viral infections, such as viral respiratory infections, comprises administering to a subject in need of such treatment an effective amount of a composition comprising a peptide agent comprising a beta thymosin peptide comprising amino acid sequence LKKTET or LKKTNT, a conservative variant thereof, or a stimulating agent that stimulates production of an LKKTET or LKKTNT peptide, or a conservative variant thereof.

### DETAILED DESCRIPTION OF THE INVENTION

Severe acute respiratory syndrome-coronavirus-2 (SARS-CoV-2) is a new β-coronavirus responsible for the pandemic viral pneumonia known as COVID-19. COVID-19 causes a wide spectrum of clinical manifestations, ranging from asymptomatic or paucisymptomatic forms (with cough, fever, myalgia, and malaise) to full-blown viral pneumonia, which can lead to acute respiratory distress syndrome (ARDS). Three phenotypes are observed in COVID-19 patients, indicating three stages of the infection's progression and extent: (i) “mild” (benign infection: 80%) in patients with minor and nonspecific symptoms who will not progress to a more severe disease; (ii) “moderate” (overt pneumonia with or without hypoxia and localized inflammation: 15%) in patients requiring hospitalization; and (iii) “severe” (systemic hyperinflammation and ARDS: 5%) in patients who require critical care management and at risk of fatal outcome (1-2%). Lung injury directly induced by the virus remains poorly explained. Patients with high viral loads and long virus-shedding periods are at higher risk of severe COVID-19.

Current pipeline approaches to treat viral respiratory infections including in COVID-19 patients involve vaccine development, blocking viral replication, and prevention of viral entry into the lung and other tissues. There is an urgent need for novel therapies to prevent the death of approximately 80% of those patients on ventilators, to reduce the time on the ventilator, and/or to prevent long term lung and other tissue damage from the ARDS cytokine, chemokine and/or bradykinin storms due to COVID-19, or other infection by a SARS virus, a coronavirus and/or SARS-CoV-2.

Without being bound to any specific theory, actin-sequestering peptides such as thymosin beta 4 (Tβ4 or TB4) and other agents including actin-sequestering peptides or peptide fragments containing amino acid sequence LKKTET or LKKTNT or conservative variants thereof, are useful for treating viral infections such as viral respiratory infections in patients suffering therefrom.

In accordance with one embodiment, the invention is a method of treating viral respiratory infections, in a subject, comprising administering to a subject in need of such treatment an effective amount of a composition comprising a peptide agent, which may be a polypeptide comprising amino acid sequence LKKTET or LKKTNT, or a conservative variant thereof, e.g., Thymosin β4, and/or Tβ4 isoforms, analogues or derivatives, including KLKKTET, LKKTETQ, N-terminal variants of Tβ4, C-terminal variants of Tβ4and antagonists of Tβ4. The invention also may utilize oxidized Tβ4. In accordance with other embodiments, the agent is other than thymosin beta 4 or other than oxidized Tβ4.

Compositions which may be used in accordance with the present invention include beta thymosin peptide agents such as Thymosin β4 (Tβ4), and/or Tβ4 isoforms, analogues or derivatives, including oxidized Tβ4, N-terminal variants of Tβ4, C-terminal variants of Tβ4 and antagonists of Tβ4, polypeptides or peptide fragments comprising or consisting essentially of the amino acid sequence LKKTET or conservative variants thereof. International Application Serial No. PCT/US99/17282, incorporated herein by reference, discloses isoforms of T4 which may be useful in accordance with the present invention as well as amino acid sequence LKKTET and conservative variants thereof, which may be utilized with the present invention. International Application Serial No. PCT/GB99/00833 (WO 99/49883), incorporated herein by reference, discloses oxidized Thymosin β4 which may be utilized in accordance with the present invention. Although the present invention is described primarily hereinafter with respect to Tβ4 and Tβ4 isoforms, it is to be understood that the following description is intended to be equally applicable to amino acid sequence LKKTET or LKKTNT, peptides and fragments comprising or consisting essentially of LKKTET or LKKTNT, conservative variants thereof having viral infection-inhibiting activity, and/or Tβ4 isoforms, analogues or derivatives, including N-terminal variants of Tβ4, C-terminal variants of Tβ4 and antagonists of Tβ4. The invention also may utilize oxidized Tβ4.

In one embodiment, the invention provides a method of treating viral respiratory infections, in a subject, comprising administering to a subject in need of such treatment an effective amount of a composition comprising a peptide agent comprising amino acid sequence LKKTET or LKKTNT, a conservative variant thereof, or a stimulating agent that stimulates production of an LKKTET or LKKTNT peptide, or a conservative variant thereof.

The present disclosure includes using a synthetic form of the naturally-occurring thymosin beta 4 protein (Tβ4) for protective activities, including anti-inflammation, anti-apoptosis, and tissue repair/regeneration to treat/prevent the lung and other tissue damage observed in COVID-19 patients and other related respiratory virus patients as well as those patients having pneumonia. The present disclosure aligns with the BAA Influenza and Emerging Infectious Disease Therapeutics (Area #9 and sub area #9.3 Immunomodulators or Therapeutics Targeting Lung Repair) to define a therapeutic for lung damage and with the PHEMCE emergency preparedness for medical countermeasures to provide a treatment for lung damage that can be stockpiled if needed. Tβ4 has shown long-term stability in the lyophilized state and can easily be reconstituted in physiological liquid (highly soluble).

Tβ4 is a small 43 amino acid, naturally-occurring, protective and regenerative molecule found in all tissues/cells and fluids in the body. It has multiple biological activities, which include protection from tissue damage and inhibition of inflammation, apoptosis (improved cell survival), decreased oxidative stress, down-regulation of inflammatory chemokines/cytokines, promotion of cell migration, blood vessel formation, reduction of scar formation, and stem cell recruitment and maturation. Active sites for many of these activities have been defined in short Tβ4 sequence peptides, two of which are naturally-occurring in the blood and wound fluid.

In one aspect, the present disclosure includes using beta thymosin peptides such as Tβ4 to decrease inflammation and thus prevents swelling, reducing cell death, i.e.

promoting cell survival and reduces scar formation that helps to maintain survival and tissue function. Further, the present disclosure includes thymosin peptides such as Tβ4 promoting new blood vessel formation (angiogenesis). In one aspect, the present disclosure includes forming new blood vessels are needed to supply oxygen and nutrients to the tissues for maintenance, to promote growth, and to remove waste products.

In one aspect, the present disclosure includes providing protective and restorative effects using a beta thymosin peptide such as Tβ4 on lung and other tissue including reducing or halting of the inflammatory process and reducing leukocytes), reducing histological evidence of lung and other tissue injury, and decreasing collagen content in the lung and other tissue.

In one aspect, the present disclosure includes blocking, inhibiting, controlling and/or reducing undesired cytokine release including cytokine release syndromes in the lung and other tissue. Cytokines and chemokines may include but are not limited to TNF-α, IL-1β, IL-1Rα, sIL-2Rα, IL-6, IL-10, IL-17, IL-18, IFN-γ, MCP-3, M-CSF, MIP-1a, G-CSF, IP-10 and MCP-1.

In one aspect, the present disclosure includes blocking IL17-producing cells in the blood using a beta thymosin peptide such as Tβ4.

In one aspect, the present disclosure includes using beta thymosin peptides such as TB4 blocking hypercytokinemia usinga.

In one aspect, the present disclosure includes blocking IL-1β, IL-1Ra, IL-6, IL-7, IL-10, IP-10, and TNF-α using a beta thymosin peptide such as Tβ4.

In one aspect, the present disclosure includes ameliorating, reducing and/or down-regulating a bradykinin inflammatory storm in a subject infected with at least one of a SARS virus, a coronavirus or SARS-CoV-2, using beta thymosin peptides such as TB4.

In one aspect, the present disclosure includes ameliorating, reducing and/or down-regulating a cytokine storm in a subject infected with at least one of a SARS virus, a coronavirus or SARS-CoV-2, using beta thymosin peptides such as TB4.

In one aspect, the present disclosure includes ameliorating, reducing and/or down-regulating a chemokine storm in a subject infected with at least one of a SARS virus, a coronavirus or SARS-CoV-2, using beta thymosin peptides such as TB4.

In one aspect, the present disclosure includes ameliorating, reducing and/or down-regulating at least one of a bradykinin inflammatory storm, a cytokine storm, and/or a chemokine storm, in a subject infected with at least one of a SARS virus, a coronavirus or SARS-CoV-2, using beta thymosin peptides such as TB4.

In one aspect, a beta thymosin peptide can administered through any suitable method, such as injection, intravenously, subcutaneously, intramuscularly, by ingestion, sub-lingually, or directly applied to affected tissue, e.g., as a solution, suspension, mist, aerosol, or the like.

In one aspect, a method of treating a patient infected with at least one of a SARS virus, a coronavirus or SARS-CoV-2, comprises administering an effective amount of a beta thymosin peptide comprising amino acid sequence LKKTET or LKKTNT.

In one aspect, a beta thymosin peptide is administered by injection, intravenously, subcutaneously, intramuscularly, by ingestion, sub-lingually, or directly applied to affected tissue.

In one aspect, a beta thymosin peptide is administered as a solution, suspension, mist or aerosol.

In one aspect, a beta thymosin peptide is administered to achieve a desired concentration of said peptide by iv injection.

In one aspect, a method of at least one of ameliorating, reducing or down-regulating at least one of a bradykinin inflammatory storm, a cytokine storm, or a chemokine storm, in a subject infected with at least one of a SARS virus, a coronavirus or SARS-CoV-2, comprises administering an effective amount of a beta thymosin peptide comprising amino acid sequence LKKTET or LKKTNT.

In one aspect, a method of enhancing healing of at least one of damaged or injured lung tissue, pulmonary tissue, respiratory tissue, heart tissue, kidney tissue, liver zo tissue, or blood vessels comprises administering an effective amount of a beta thymosin peptide comprising amino acid sequence LKKTET or LKKTNT.

In one aspect, lung morbidity is reduced using beta thymosin peptides such as TB4.

In one aspect, administration is for 6, 8, 10, 12, 24, 36, 48 hours, or longer using beta thymosin peptides such as TB4.

In one aspect, a dosage is within the range of 0.01 mg/kg body weight/day to 50 mg/kg body weight/day using beta thymosin peptides such as TB4.

In one aspect, an effective amount of the peptide may be a range of about 0.1-40 mg using beta thymosin peptides such as TB4.

In one aspect, a unit dosage of the peptide may be administered 1-6 times per day using beta thymosin peptides such as TB4.

In one aspect, a daily amount of the peptide may be about 0.1-40 mg, about 1-30 mg, about 5-20 mg, about 1-15 mg, and any range disclosed or contemplated herein may administered using beta thymosin peptides such as TB4.

In one aspect, the present disclosure includes blocking, reducing or preventing lymphopenia or lymphocyte exhaustion using a beta thymosin peptide such as Tβ4.

In one aspect, the present disclosure includes blocking neutrophil-recruiting mediators (CXCL8, CXCL1, CXCL2, CXCL10, CCL2, CCL7) and other attractants of monocytes and immune cells (CXCL6, CXCL11, CCL2, CCL3, CCL4, CCL7, CCL8, CCL20) using a beta thymosin peptide such as Tβ4.

In one aspect, the present disclosure includes blocking lung infiltration by monocytes, macrophages and neutrophils using a beta thymosin peptide such as Tβ4.

In one aspect, the present disclosure includes reducing lung oxidative stress and inflammation by administration of a beta thymosin peptide such as Tβ4.

In one aspect, the present disclosure includes treating lung fibrosis by administration of a beta thymosin peptide such as Tβ4.

In one aspect, the present disclosure includes using a beta thymosin peptide such as Tβ4 in the protecting, repairing and regenerating the lungs of COVID19 patients resulting in increased survival, reduced time on the ventilator, and reduced long-term damage. In one aspect, the present disclosure includes providing a safe and effective therapy for such patients including in various fragile patient populations (elderly, diabetic, immunocompromised, and pediatric).

SARS-CoV-2, other SARS viruses, and other coronaviruses not only damage lung tissue, but may also damage heart tissue, kidney tissue, liver tissue, blood vessels, and other tissues. In some aspects, SARS-CoV-2, other SARS viruses, and other coronaviruses may induce some of the same or similar symptoms as seen in Kawasaki disease in children. Such symptoms may include autoimmune-like pathologies associated with inflammatory chemokine storm, cytokine storm, and/or bradykinin storm. Beta thymosin peptides such as TB4 significantly down regulate these molecules. Without being bound to any particular theory, beta thymosin peptides such as TB4 reduce fibrosis and scarring in affected tissues. Beta thymosin peptides such as TB4 have wound healing properties that accelerate healing of affected tissues. Beta thymosin peptides such as TB4 may restore immune balance. Beta thymosin peptides such as TB4 may reduce Kawasaki-like symptoms in the inflamed blood vessels and other organs of patients infected with SARS-CoV-2, other SARS viruses, and/or other coronaviruses.

The present disclosure includes delivering a beta thymosin peptide such as Tβ4 in solution directly into the lung via endotracheal intubation in patients that have been put on mechanical ventilation as a result of COVID-19. In one aspect, the disclosure will reduce, inhibit, prevent, or eliminate the overwhelming inflammatory response and morbidity associated with the virus in the lungs and subsequent amount of time on ventilation. A beta thymosin peptide such as Tβ4 may potentially reduce time of hospitalization and any long-term damage, such as scarring, to the lung.

In one aspect, the Beta thymosin peptide such as Tβ4 is lyophilized in individual glass containers per each patient. The hospital pharmacy may reconstitute doses of Tβ4 at a concentration of 0.1% (isotonic saline), per 3 mL dose. Each vial will thus contain approximately 10 doses per patient. Tβ4 may be administered intratracheally via ETT in situ as soon as possible but no later than 3 hours after initial intubation q 8 h for 3 days. A beta thymosin peptide such as Tβ4 may be administered through the endotracheal intubation tube directly into the lung. The primary endpoints may be duration of time on the ventilator and % survival.

Administration of a beta thymosin peptide such as Tβ4 may provide direct application of a beta thymosin peptide such as Tβ4 to the most active site of pulmonary inflammation during the cytokine storm phase of this disease and may minimize risk to the healthcare staff given from infection with SARS-CoV-2 and related respiratory diseases. In one aspect, the present method of administering a beta thymosin peptide such as Tβ4ameliorates the lung tissue reactions, improves survival, decreases time on a ventilator, and prevents long-term damage to lung tissue.

The administration may be for 6, 8, 10, 12, 24, 36, 48 hours, or longer, using a beta thymosin peptide such as Tβ4. Dosages may be within the range of 0.01 mg/kg body weight/day to 50 mg/kg body weight/day. According to one embodiment, the effective amount may be a range of about 0.1-40 mg. The effective amount dosage may be a unit dosage. The unit dosage may be administered 1-6 times per day. A daily amount may include about 0.1-40 mg, about 1-30 mg, about 5-20 mg, about 1-15 mg, and any range disclosed or contemplated herein.

In one aspect, a patient is treated by contacting the affected tissue with an effective amount of a composition which contains a peptide agent as described herein. Examples of direct administration include, for example, contacting the tissue, by direct application or inhalation, with a solution, lotion, salve, gel, cream, paste, spray, suspension, dispersion, hydrogel, ointment, or oil comprising a peptide agent as described herein. Systemic administration includes, for example, intravenous, intraperitoneal, intramuscular injections of a composition containing a peptide agent as described herein, in a pharmaceutically acceptable carrier such as water for injection.

Peptide agents for use in the invention, as described herein, may be administered in any effective amount. For example, a peptide agent as described herein may be administered in dosages within the range of about 0.0001-1,000,000 micrograms, or in amounts within the range of about 0.1-5,000 micrograms, or within the range of about 1-100 micrograms.

A composition in accordance with the present invention can be administered daily, every other day, every other week, every other month, etc., with a single application or multiple applications per day of administration, such as applications 2, 3, 4 or more times per day of administration, using a beta thymosin peptide such as Tβ4. Regimens of administration can take place for one week, two weeks, three weeks, four weeks, or more.

1. A method of treating a patient infected with at least one of a SARS virus, a coronavirus or SARS-CoV-2, comprising administering an effective amount of a beta thymosin peptide comprising amino acid sequence LKKTET or LKKTNT.

2. The method of claim 1 wherein the peptide is administered by injection, intravenously, subcutaneously, intramuscularly, by ingestion, sub-lingually, or directly applied to affected tissue.

3. The method of claim 2 wherein the peptide is administered as a solution, suspension, mist or aerosol.

4. The method of claim 3 wherein the peptide is administered to achieve a desired concentration of said peptide in the patient's lung.

5. The method of claim 3, by administering said peptide to achieve a desired concentration of said peptide by iv injection.

6. The method of claim 1, wherein lung morbidity is reduced.

7. The method of claim 1, wherein administration is for 6, 8, 10, 12, 24, 36, 48 hours, or longer.

8. The method of claim 1, wherein dosage is within the range of 0.01 mg/kg body weight/day to 50 mg/kg body weight/day.

9. The method of claim 1, wherein the effective amount may be a range of about 0.1-40 mg.

10. The method of claim 1, wherein a unit dosage may be administered 1-6 times per day.

11. The method of claim 1, wherein a daily amount of about 0.1-40 mg, about 1-30 mg, about 5-20 mg, about 1-15 mg is administered.

12. The method claim 1, wherein the peptide is TB4.

13. A method of at least one of ameliorating, reducing or down-regulating at least one of a bradykinin inflammatory storm, a cytokine storm, or a chemokine storm, in a subject infected with at least one of a SARS virus, a coronavirus or SARS-CoV-2, comprising administering an effective amount of a beta thymosin peptide comprising amino acid sequence LKKTET or LKKTNT.

14. The method of claim 13 wherein the peptide is administered to achieve a desired concentration of said peptide in the subject's lung.

15. The method of claim 13, wherein the effective amount is in a range of about 0.1-40 mg.

16. The method claim 13, wherein the peptide is TB4.

17. A method of enhancing healing of at least one of damaged or injured lung tissue, pulmonary tissue, respiratory tissue, heart tissue, kidney tissue, liver tissue, or blood vessels in a subject infected with at least one of a SARS virus, a coronavirus or SARS-CoV-2, comprising administering an effective amount of a beta thymosin peptide comprising amino acid sequence LKKTET or LKKTNT.

18. The method of claim 17 wherein the peptide is administered to achieve a desired concentration of said peptide in the subject's lung.

19. The method of claim 17, wherein the effective amount is in a range of about 0.1-40 mg.

20. The method of claim 17, wherein the peptide is TB4.


SEQUENCE LISTING (4 sequences)

SEQ ID NO: 1 - protein, 6 aa, artificial sequence
  beta thymosin peptide
LKKTET

SEQ ID NO: 2 - protein, 6 aa, artificial sequence
  beta thymosin peptide
LKKTNT

SEQ ID NO: 3 - protein, 7 aa, artificial sequence
  Thymosin beta 4, and/or T beta 4 isoforms, analogues or derivatives
KLKKTET

SEQ ID NO: 4 - protein, 7 aa, artificial sequence
  Thymosin beta 4, and/or T beta 4 isoforms, analogues or derivatives
LKKTETQ